(19) United States
(12) Patent Application Publication
(10) Pub. No.: US 2020/0107849 A1
(43) Pub. Date: Apr. 9, 2020

(54) ACTUATING SCALPEL DEVICE

(71) Applicants: Chris Salvino, SCOTTSDALE, AZ (US); Keir Hart, Lafayette, CO (US); Mark J. Huseman, Broomfield, CO (US)

(72) Inventors: Chris Salvino, SCOTTSDALE, AZ (US); Keir Hart, Lafayette, CO (US); Mark J. Huseman, Broomfield, CO (US)

(21) Appl. No.: 16/592,723

(22) Filed: Oct. 3, 2019

Related U.S. Application Data
(60) Provisional application No. 62/742,529, filed on Oct. 8, 2018.

(51) Int. Cl. A61B 17/3211; A61B 17/3209; A61M 27/00

(52) U.S. Cl. CPC A61B 17/3211; A61B 17/32093; A61B 2017/00367; A61B 2017/32113; A61M 27/00

(57) ABSTRACT

An actuating scalpel device that is adapted to deploy a curved scalpel blade in an elliptical or circular pathway is described in detail. The actuating scalpel generally comprises a scalpel blade that possesses a curved cutting edge, a probe tip housing that fully contains said scalpel blade when in a retracted position, a slot in said housing through which said curved cutting edge is adapted to extend and a driving member connected to said scalpel blade. The linkages are configured to move said scalpel blade in an elliptical path when said scalpel blade is deployed to extend outside of said probe tip housing.

### CROSS-REFERENCE TO RELATED APPLICATIONS

This application claims priority to and the benefit of U.S. provisional Patent Application No. 62/742,529, entitled: AN ACTUATING SCALPEL DEVICE, filed on Oct. 8, 2018.

### FIELD OF THE INVENTION

The present embodiments are directed to an actuating scalpel device with applications in a chest tube insertion device.

### DESCRIPTION OF RELATED ART

The lungs are surrounded by a pleural sac made up of two membranes, the visceral and parietal pleurae. The parietal pleura lines the thoracic wall, and the visceral pleura surrounds the lung. The pleural space is a potential space between these two layers of pleurae. It contains a thin layer of serous pleural fluid that provides lubrication for the pleurae and allows the layers of pleurae to smoothly slide over each other during respiration. In abnormal circumstances the pleural space can fill with air and certain types of fluids not normally present; many times these fluids and/or air require drainage.

In the industrialized world, trauma is the leading cause of death in males under the age of forty. In the United States, chest injuries are responsible for one-fourth of all trauma deaths. Many of these fatalities could be prevented by early recognition of the injury followed by prompt management. Some traumatic chest injuries require quick placement of chest tubes to drain out air and/or fluids (such as blood) from the chest cavity. With respect to non-trauma patients; some medical patients also require the placement of chest tubes to drain out air and/or fluids.

Several techniques are currently used to insert a chest tube, each of which involves a relatively lengthy manual procedure that requires knowledge and experience. The most common technique involves surgical preparation and draping at the site of the tube insertion (usually at the nipple level-fifth intercostal space, anterior to the mid-axillary line on the affected side), administering of local anesthesia to the insertion site, and making a 2-4 cm vertical incision. A clamp is inserted through the incision and spread tearing muscle and tissue until a tract large enough to accept a finger is created. Next, the parietal pleura is punctured. One way is with the tip of a clamp, and the physician, on occasion, places a gloved finger into the incision to confirm the presence of a free pleural space locally. Next, the proximal end of the chest tube is advanced through the incision into the pleural space. As the chest tube is inserted, it is sometimes directed posteriorly and superiorly towards the apex of the lung or elsewhere in the chest cavity. Once in position, the goal is for the chest tube to drain the pleural space of both air and/or fluids such as blood.

Once the chest tube is appropriately in place (the tube is connected to a water-seal apparatus or to another one-way valve in order to clear air and/or fluids (such as blood, infection, a transudate) from the pleural space. The tube is sutured to the skin, dressing is applied, and the tube is taped to the chest.

Insertion of a chest tube using this standard technique can require more than 15 minutes to accomplish by a physician, requires extensive medical training to be performed properly and can be extremely painful as it is a difficult area to anesthetize due to the intercostal nerve that runs on the bottom of every rib. Further, while performing the procedure, the physician must attend to the patient receiving the chest tube and thus is precluded from attending to other patients.

Insertion of chest tubes using the standard technique is considered an open system. In other words, fluids and/or air within the thoracic cavity can exit the chest tube into the atmosphere as the tube is being inserted into the chest and expose the medical staff to skin contact and aerosolized/inhaled pathogens from the patient with known or unknown pathogens.

FIG. 1 depicts a prior art chest tube insertion gun 100 described in U.S. Pat. No. 7,811,293. This chest tube insertion gun 100 includes a housing 105, a handle 110 with the trigger 125, a probe tip 130 having a circular cutting tip 135 at the distal end thereof, a circular cross-sectioned cannula 140, a circular cross sectioned chest tube 145. The circular cutting tip 135 rotates outside of the distal end up to a 90° angle of rotation (rotation angle) from its neutral position before rotating back to its neutral position. The circular cutting tip 135 is also able to rotate a small negative angle from its neutral position in order to retract inside of the distal end of the probe tip 130. The rotation angle works well for the circular cross-sectioned cannula 140.

It is to innovations related to this subject matter that the claimed invention is generally directed.

### SUMMARY OF THE INVENTION

The present embodiments are directed to an actuating scalpel device with applications in a chest tube insertion device. The actuating scalpel device is adapted and arranged or otherwise configured to deploy a curved scalpel blade in an elliptical or circular pathway.

Certain embodiments of the present invention contemplate an actuating scalpel device comprising: a scalpel blade that possesses a curved cutting edge; a probe tip housing that fully contains said scalpel blade when in a retracted position; a slot in said housing through which said curved cutting edge is adapted to extend; a driving member connected to said scalpel blade and configured to move said scalpel blade in an elliptical path when said scalpel blade is deployed to extend outside of said probe tip housing.

Yet other certain embodiments of the present invention contemplate a method comprising providing an actuating scalpel device that possesses a scalpel blade having a curved cutting edge, a probe tip housing that fully contains said scalpel blade when in a retracted state, a slot in a distal end of said probe tip housing; deploying at least said curved cutting edge to extend outside of said probe tip housing via said slot, said scalpel blade moving in an elliptical path defined by a single point on said curved cutting edge traveling from a) when said scalpel blade is in said retracted position to b) when deployed to move outside of said probe tip housing to c) back to said retracted position.

While other certain embodiments of the present invention contemplate an actuating scalpel device comprising: a scalpel blade having a noncircular curved cutting edge; a housing that fully contains said scalpel blade when said scalpel blade is in a retracted state; a slot in a distal end of said housing through which at least said curved cutting edge is adapted to extend; a drive member connected to said scalpel blade; means for actuating said drive member; and means for driving said scalpel blade to extend out of said slot in an elliptical pathway via said drive member.

### BRIEF DESCRIPTION OF THE DRAWINGS

FIG. 1 illustratively depicts a prior art chest tube insertion gun;

FIG. 2A illustratively depicts a side view of an actuator scalpel consistent with embodiments of the present invention;

FIG. 2B illustratively depicts the side view of the actuator scalpel of FIG. 1A with the insertion gun body housing removed to view the internal mechanism consistent with embodiments of the present invention;

FIG. 3A illustratively depicts a side view of an actuator scalpel embodiment possessing a larger scalpel and probe tip consistent with embodiments of the present invention;

FIG. 3B illustratively depicts a front isometric view of the actuator scalpel embodiment of FIG. 3A;

FIG. 3C illustratively depicts a front view of the scalpel tip from the actuator scalpel embodiment of FIG. 3A;

FIG. 4 illustratively depicts a side view of a scalpel blade consistent with embodiments of the present invention;

FIG. 5A illustratively depicts the actuator scalpel with many of the internal components revealed including the handle in a fully extended configuration consistent with embodiments of the present invention;

FIG. 5B is a side view drawing of the actuator scalpel of FIG. 5A but without the handle consistent with embodiments of the present invention;

FIG. 5C is an enlarged side view drawing of the scalpel blade of FIG. 5A consistent with embodiments of the present invention;

FIG. 6A is a side view drawing of the actuator scalpel depicting initial deployment of the scalpel blade consistent with embodiments of the present invention;

FIG. 6B is an enlarged side view drawing of the scalpel blade of FIG. 6A consistent with embodiments of the present invention;

FIG. 7A is a side view drawing of the actuator scalpel depicting full deployment of the scalpel blade consistent with embodiments of the present invention;

FIG. 7B is an enlarged side view drawing of the scalpel blade of FIG. 7A consistent with embodiments of the present invention;

FIG. 8A is a side view drawing of the actuator scalpel depicting the position of the scalpel blade as it returns to the probe tip housing consistent with embodiments of the present invention;

FIG. 8B is an enlarged side view drawing of the scalpel blade of FIG. 8A consistent with embodiments of the present invention; and

FIGS. 9A and 9B illustratively depict the pathway of a single point on the curved cutting edge in various stages of scalpel blade deployment consistent with embodiments of the present invention.

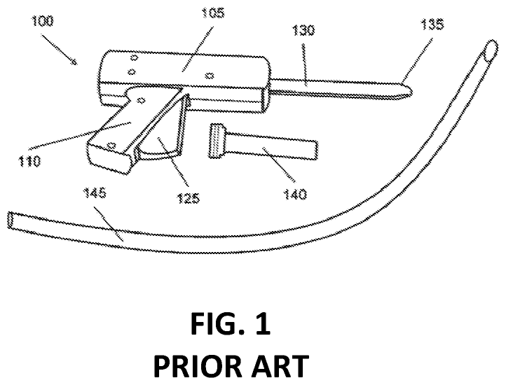

### DETAILED DESCRIPTION

Initially, this disclosure is by way of example only, not by limitation. Thus, although the instrumentalities described herein are for the convenience of explanation, shown and described with respect to exemplary embodiments, it will be appreciated that the principles herein may be applied equally in other types of situations involving similar uses of an actuator scalpel. In what follows, similar or identical structures may be identified using identical callouts.

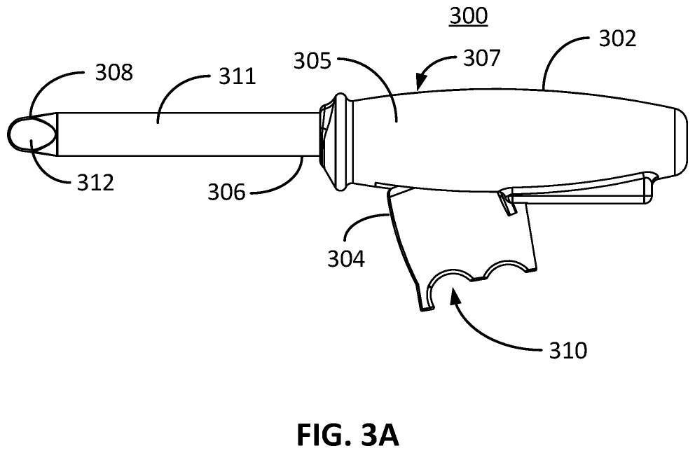

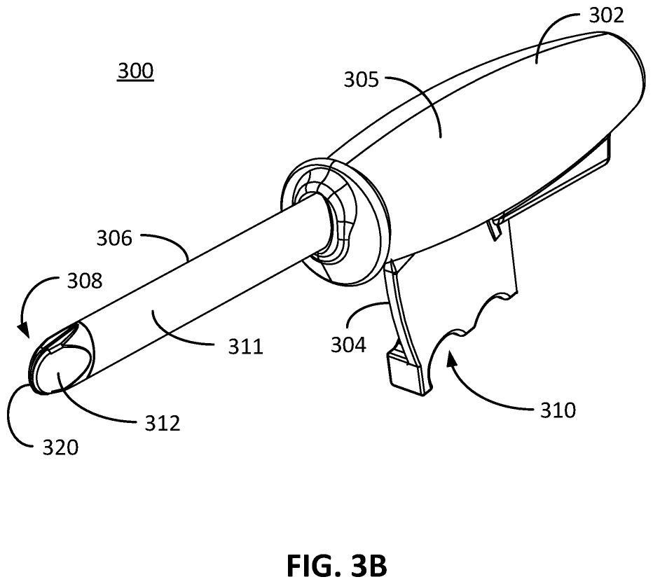

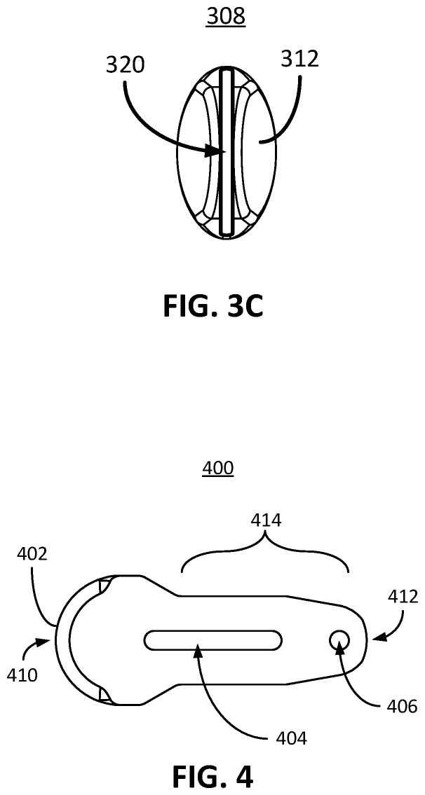

In essence as viewed with reference to FIGS. 3B and 4, described herein are actuating scalpel device embodiments (such as scalpel device 300) that in one aspect of the present invention focuses on a probe tip region. The probe tip region 308 possesses a probe tip housing 312 that fully contains a scalpel blade 400 when in a retracted or otherwise unused/starting position. There is a probe tip slot 320 that passes through or otherwise makes an opening in the probe tip housing 320 through which the curved cutting edge of the scalpel blade 400 is adapted to extend in both an elliptical motion and a rotating/slicing motion due to the configuration of the driving mechanism and accommodating features of the cutting blade 400.

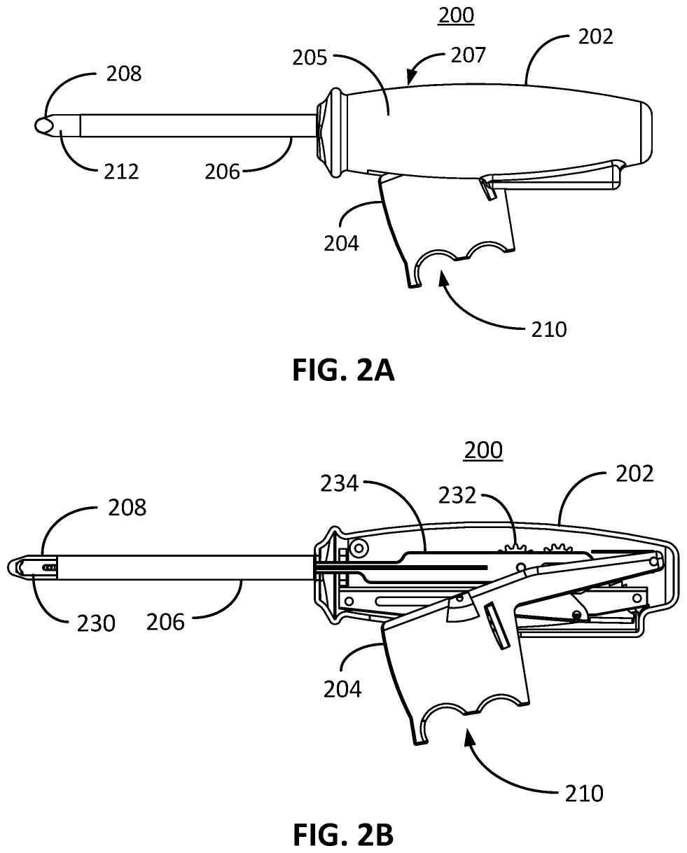

FIG. 2A illustratively depicts a side view of an actuator scalpel consistent with embodiments of the present invention. FIG. 2B illustratively depicts a side view of the actuator scalpel 200 of FIG. 2A revealing some of the internal components inside of the handle body 202 and the probe tip 208 consistent with embodiments of the present invention. As shown in both figures, the actuator scalpel 200 possesses a handle body 202, a trigger 204, a probe 206, and a probe tip 208. FIG. 2B illustratively shows the actuator scalpel 200 with a body housing (panel) 205 and a probe tip housing (panel) 212 removed. As will be discussed in more detail, infra, a drive arm member 234 drives/moves a curved scalpel 230 by way of rotating gears 232 when the trigger 204 is actuated. In operation, the actuator scalpel 200 is gripped with an operator's (person's) palm positioned along the top of the handle body 207 and two of their fingers positioned in the finger grips 210 whereby upon squeezing the handle 204 towards the handle body 202, the scalpel 230 is made to move in a cutting motion.

FIG. 3A illustratively depicts a side view drawing of another actuator scalpel embodiment consistent with embodiments of the present invention. FIG. 3A possesses the fundamental elements described in FIGS. 2A and 2B with the exception that probe 306 and the probe tip 208 (and the associated housing 312) are sized to accommodate a larger scalpel. Accordingly, the actuator scalpel 300 comprises a handle body 302, a trigger 304, a probe 306, and a probe tip 308 showing the probe tip housing 312. As in the previous figure, the trigger 304 depicts finger grips 310 adapted to accommodate the fingers of a human hand (not shown). Shown for reference is the probe housing 311 and the body housing 305.

FIG. 3B illustratively depicts a front isometric view of the actuator scalpel 300 showing the tapered probe tip housing 312 consistent with the embodiment of FIG. 3A. In this perspective, the probe tip slot 320 is shown on the distal end of the actuator scalpel 300. The scalpel blade 400 is adapted to extend out from and beyond the probe tip housing 312 via the probe tip slot 320.

FIG. 3C illustratively depicts a front view of the scalpel tip 308 from the actuator scalpel embodiment of FIG. 3A. The probe tip slot 320 is shown in a bolder lined window in the center of the tapered probe tip housing 312.

FIG. 4 illustratively depicts a side view of a scalpel blade consistent with embodiments of the present invention. As shown, the scalpel blade 400 possesses a semicircular cutting edge 402 at the distal end 410 and a pivot aperture 406 (a hole that passes through the blade 400) at the proximal end 412. With reference to the cutting-edge 402, certain embodiments envision a noncircular curved cutting edge, a cutting edge that is a complete circle (which may or may not possess the appropriate functional pivot aperture 406 and/or the scalpel blade slot 404). A scalpel blade slot 404 that passes through the blade 400 extends between the distal end 410 in the proximal end 412 that facilitates being anchored or otherwise fixedly attached to the probe tip housing 312 via a slot pin, not shown. The scalpel blade slot 404 facilitates the scalpel blade 400 to rotate and/or move in and out of the probe tip slot 320 or some other combination while staying in plane with the main body of the scalpel blade 400. In the present embodiment, the scalpel blade 400 comprises an elongated region 414 that is narrower than the semicircular cutting edge 402 in order to accommodate the dynamic motion of the scalpel blade 400 within the probe tip housing 312. Certain embodiments envision the scalpel blade 400 being essentially comprised of a unitary plate thereby defining a plane (obviously the plane excludes the cutting edge 402, chamfers and other minor surfaces of the scalpel blade 400). Other embodiments contemplate the scalpel blade 400 being composed of stainless steel or ceramic, (or other materials composed in a scalpel blade known to those skilled in the art). As depicted in the present embodiment, the semicircular cutting edge 402 (or otherwise curved cutting edge) terminates at a tapered to a thin cutting edge (i.e., a razor edge) adapted to effortlessly sliced through tissue.

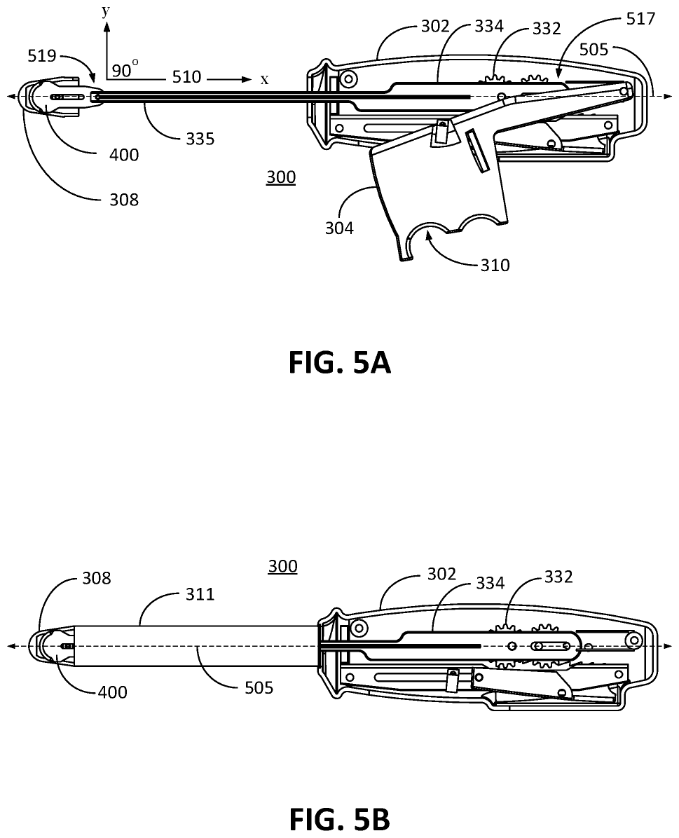

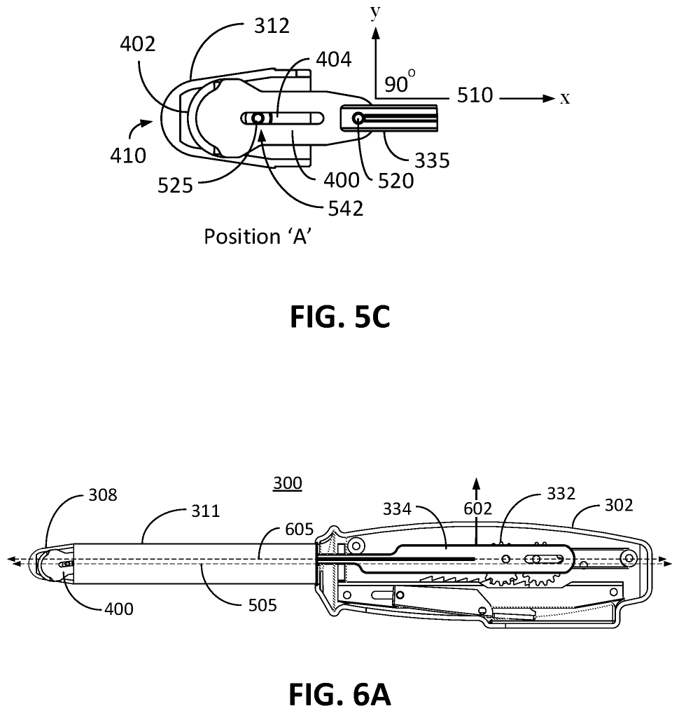

FIG. 5A illustratively depicts the actuator scalpel 300 embodiment with many of the internal components revealed consistent with embodiments of the present invention. The body housing (panel) 305, the probe tip housing (panel) 312 and the probe housing 311 are removed from the drawing to reveal some of the inner elements of the actuator scalpel 300. As shown, with the scalpel blade 400 in a fully retracted position wherein the drive arm member 334, and more specifically the drive arm rod 335 (a defined part of the drive arm member 334), is completely horizontal with respect to the scalpel blade 400 using the x/y reference 510. Accordingly, the actuator scalpel 300 is ready to be picked up whereby the trigger/handle 304 can be grasped which coupled with the gears 332 and other linkages and slots is the mean for actuating the drive member 334 to move in accordance with the embodiment shown. In other words, this is essentially the starting position/point whereby the handle 304 is not squeezed or otherwise compressed by an operator (or for that matter, even held by an operator). As shown, the driving member 334 extends from the handle body 302 at one end 517 and is connected to the scalpel blade 400 at the other end 519 (i.e., the distal end 519 of the driving member 334). The proximal end 517 and said drive member 334 distal end 519 define a drive member reference line 505, said drive member reference line 505 remaining parallel to when said drive member reference line 505 is in said retracted position as shown no matter what other positions the drive member 334 is in (i.e., while deploying the scalpel blade outside of the probe tip housing 400) as will be readily obvious in the later figures.

FIG. 5B is a side view drawing of the actuator scalpel 300 of FIG. 5A but without the handle 304 consistent with embodiments of the present invention. In this view, the probe housing 311 is shown, however with the handle 304 removed from the illustration so the position of the gears 332 can be readily seen. Also, an unobstructed view of the scalpel blade 400 at the distal portion of the actuator scalpel 300 shown for convenience.

FIG. 5C is a side view drawing of the scalpel blade 400 in a fully retracted position consistent with embodiments of the present invention. As shown in more detail, the entire scalpel blade 400 is completely inside of the probe tip housing 312 thereby protecting the semicircular cutting edge 402 from interacting with the outside environment (e.g., in this position an operator cannot get cut by the scalpel blade 400 when handling the actuator scalpel 300). The scalpel blade 400 is pivotally attached to the drive arm rod 335 by way of a rod pin 520. The rod pin 520 is anchored to the probe housing 311 or other internal anchor points within the probe tip region. The scalpel blade 400 is movably anchored to the probe tip housing 312 via the scalpel blade slot 404 by way of a slot pin 525. The scalpel blade slot 404 and slot pin 525 arrangement facilitates movement of the scalpel blade 400 2-dimensionally (i.e., in a plane) in both the x-y directions defined by the frame of reference 510.

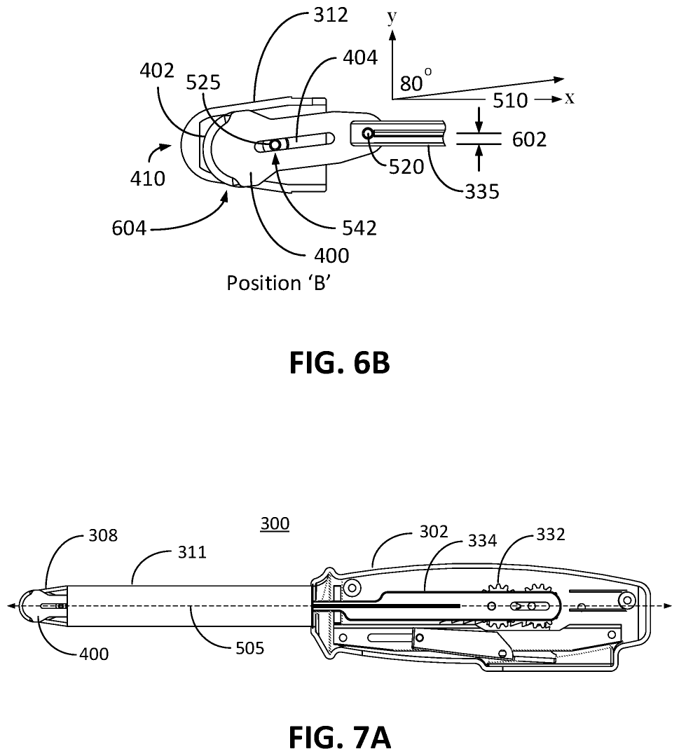

FIG. 6A is a side view drawing of the actuator scalpel 300 without the handle 304 with deployment of the scalpel blade just initiated consistent with embodiments of the present invention. In this view, the probe housing 311 is shown with the handle 304 removed from the illustration so one can readily see the position of the gears 332 and an unobstructed view of the distal portion of the drive arm member 334. In this figure, though not shown, the trigger 304 is initially squeezed thereby deploying the scalpel blade 400 start its path outside of the slot 320. The drive arm member 334 is moved upward in the direction of the arrow 602 by way of the gears 332 with a vertical displacement illustrated by the space from the baseline 505 to the displaced position 605. In the present embodiment, the drive arm member 334 remains essentially horizontal while positionally moved upwards 602. Or in other words, the drive member reference line 605 stays parallel to retracted position reference line 505.

As shown in FIG. 6B, this second position, position ‘B’, is of the scalpel blade 400 being initially deployed to extend outside of the probe tip housing 312 via the slot 320. By moving the drive arm member 334 upwards the drive arm rod 335 is displaced from the center of the slot pin 525 a vertical distance 602 to the center of the rod pin 520. Accordingly, as the drive arm rod 335 is moved in an upward direction, the scalpel blade 400 freely pivots about the pivot point 406 of the rod pin 520 and slidingly rotates along the scalpel blade slot 404 via the cooperating slot pin 525. In this depiction, the slot pin 525 is located closer to the distal end of the scalpel blade 400 within the scalpel blade slot 404 at position 542. Also shown is the scalpel cutting edge 402 is just moving outside of the distal probe tip housing 312 shown by arrow 604. In this second position ‘B’, the scalpel blade 400 is tipped is an angle of about −10° as shown by the x/y reference 510.

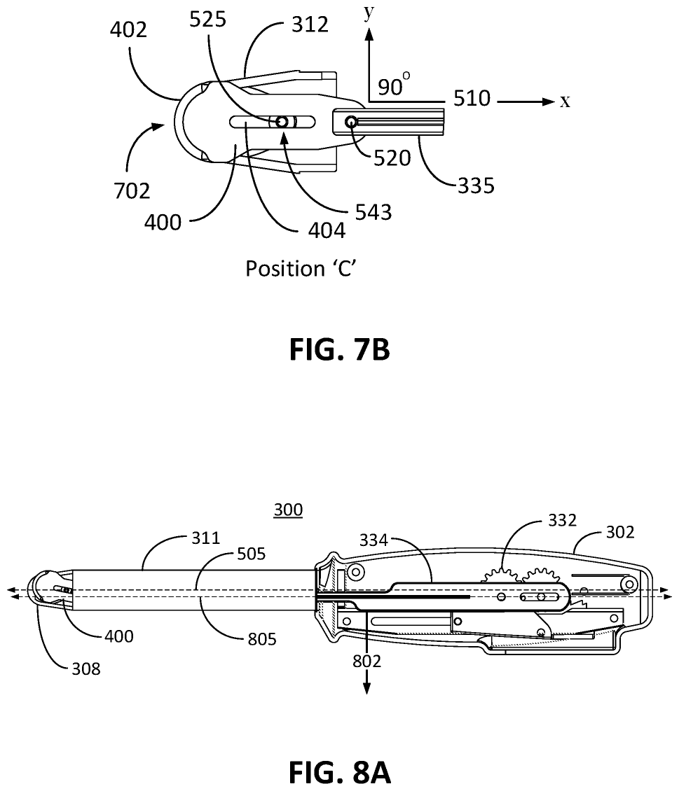

FIG. 7A is a side view drawing of the actuator scalpel 300 without the handle 304 with the scalpel blade fully deployed consistent with embodiments of the present invention. In this view, the probe housing 311 is shown with the handle 304 removed from the illustration so one can readily see the position of the gears 332 and an unobstructed view of the distal portion of the drive arm member 334. In this figure, the trigger 304 is squeezed to a point where the scalpel blade 400 is fully extended outside of the slot 320. As shown, the drive arm member 334 is moved in a position that is in line with the baseline 505 by way of the gears 332. Once again, in the present embodiment, the drive arm member 334 remains essentially horizontal while positionally moved outward via the gears 332.

As shown in FIG. 7B, this third position, position ‘C’, is of the scalpel blade 400 being fully deployed to extend outside of the probe tip housing 312 via the slot 320. The drive arm member 334 is moved back down at the centerline position 505 and outward distally. Accordingly, as the drive arm rod 335 is moved in-line with the centerline 505, the scalpel blade 400 continues to freely pivot about the pivot point of the rod pin 520 thereby slidingly rotating the scalpel blade 400 to a neutral position that is fully extended via the slot pin 525 that cooperates with the scalpel blade slot 404. In this depiction, the slot pin 525 is located closer to the proximal end of the scalpel blade 400 within the scalpel blade slot 404 at position 543. Also shown is the scalpel cutting edge 402 fully extended outside of the distal probe tip housing 312 shown by arrow 702. In this third position ‘C’, the scalpel blade 400 is place within angle of 0° as shown by the x/y reference 510.

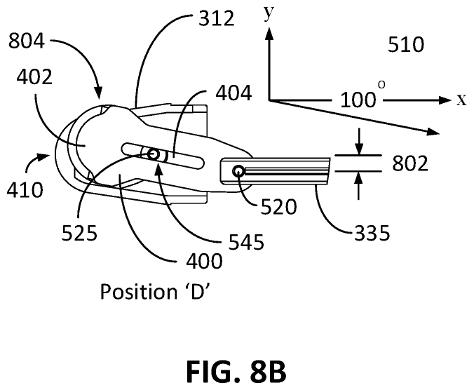

FIG. 8A is a side view drawing of the actuator scalpel 300 without the handle 304 with deployment of the scalpel blade on its journey back to the fully retracted position consistent with embodiments of the present invention. In this view, the probe housing 311 is shown with the handle 304 removed from the illustration so one can readily see the position of the gears 332 and an unobstructed view of the distal portion of the drive arm member 334. As shown here, the drive arm member 334 is moved downward in the direction of the arrow 802 with a vertical displacement illustrated by the space from the baseline 505 to the displaced position 805. In the present embodiment, the drive arm member 334 remains essentially parallel to the baseline 505 while positionally moved downwards 802.

As shown in FIG. 8B, this fourth position, position ‘D’, is of the scalpel blade 400 returning to the fully retracted position within the probe tip housing 312. By moving the drive arm member 334 downwards, the drive arm rod 335 is displaced a vertical distance 802 from the center of the slot pin 525 to the center of the rod pin 520. Accordingly, as the drive arm rod 335 is moved in a downward direction, the scalpel blade 400 freely pivots about the pivot point of the rod pin 520 and slidingly rotates via the slot pin 525 that cooperates with the scalpel blade slot 404. In this depiction, the slot pin 525 is once again located closer to the distal end of the scalpel blade 400 within the scalpel blade slot 404 at position 545. Also shown is the scalpel cutting edge 402 extending outside at the top of the distal probe tip housing 312 shown by arrow 804. In this fourth position ‘D’, the scalpel blade 400 is tipped is an angle of about +10° as shown by the x/y reference 510.

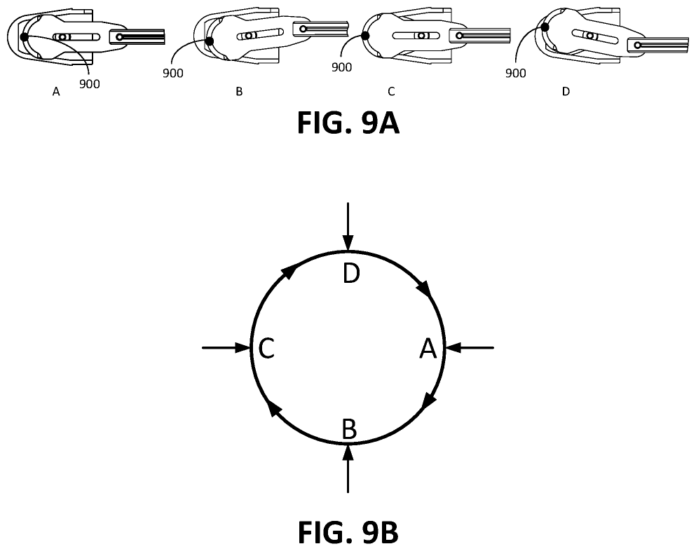

FIGS. 9A and 9B show the pathway of a single point on the curved cutting edge 402 while the scalpel blade 400 is deployed consistent with embodiments of the present invention. The four position shown in FIG. 9A are of a single point 900 on the cutting edge 402 of the scalpel blade 400. Position ‘A’ is when the scalpel blade 400 is fully retracted in the probe tip housing 312; position ‘B’ is when the scalpel blade 400 has just been deployed and is pointing downwards just outside of the probe tip housing 312 via the probe tip slot 320; position ‘C’ is when the scalpel blade 400 is fully deployed and is fully extended outside of the probe tip housing 312; position ‘D’ is when the scalpel blade 400 is tipped upwards just outside of the probe tip housing 312. FIG. 9B depicts the circular pathway the single point 900 travels when the scalpel blade 400 is deployed by squeezing the trigger 304 of the actuator scalpel 300. Certain embodiments envision that the pathway is elliptical whereby a circle is one embodiment of an ellipse. As can be appreciated, the linkage arrangement of the scalpel blade 400 (scalpel blade slot 400, the pin aperture 406 and the associated pins) serves as a means for deriving the scalpel blade to extend out of the scalpel blade slot in the elliptical pathway is shown which is all driven by the drive member 334. As can also be appreciated by viewing the different locations of the single point 900, the cutting edge 402 rotates in a slicing manner in plane with the scalpel blade 400 as a scalpel blade traverses through the elliptical, or in this case circular pathway.

It is to be understood that even though numerous characteristics and advantages of various embodiments of the present invention have been set forth in the foregoing description, together with the details of the structure and function of various embodiments of the invention, this disclosure is illustrative only, and changes may be made in detail, especially in matters of structure and arrangement of parts within the principles of the present invention to the full extent indicated by the broad general meaning of the terms in which the appended claims are expressed. For example, though an elongated semicircular cutting blade is described herein, a circular blade providing similar drivetrain related features could equally be used while still maintaining substantially the same functionality without departing from the scope and spirit of the present invention. Another example can include providing various triggers, handles, or other blade deployment structures while staying within the scope and spirit of the present invention. Yet another example can include using a motorized means for driving the scalpel blade in this desired motion that will be readily understood by a skilled artisan when using this disclosure is a guide while staying within the scope and spirit of the present invention. Further, the terms “one” is synonymous with “a”, which may be a first of a plurality.

It will be clear that the present invention is well adapted to attain the ends and advantages mentioned as well as those inherent therein. While presently preferred embodiments have been described for purposes of this disclosure, numerous changes may be made which readily suggest themselves to those skilled in the art and which are encompassed in the spirit of the invention disclosed and as defined in the appended claims.

What is claimed is:

2. An actuating scalpel device comprising:
a scalpel blade that possesses a curved cutting edge;
a probe tip housing that fully contains said scalpel blade when in a retracted position;
a slot in said housing through which said curved cutting edge is adapted to extend; and
a driving member connected to said scalpel blade at a first driving member end and an actuator at a second driving member end, said driving member connected to an elliptical drive train configured to move said scalpel blade in an elliptical path along said elliptical pivot track when said scalpel blade is deployed to extend outside of said probe tip housing.

2. The actuating scalpel device of claim 1 wherein said driving member is a rod.

3. The actuating scalpel device of claim 1 wherein said elliptical path is defined by a single point on said curved cutting edge that traverses through a) from when said scalpel blade is in said retracted position to b) when said scalpel blade is deployed to move outside of said housing to c) when said scalpel blade returns to said retracted position.

4. The actuating scalpel device of claim 3 wherein said elliptical path is a circular path.

5. The actuating scalpel device of claim 1 wherein said scalpel blade comprises an elongated element with said curved cutting-edge on a distal end of said elongated element, a pivot aperture located towards a proximal end of said elongated element, and a slotted aperture between said distal end and said proximal end.

6. The actuating scalpel device of claim 5 wherein said driving member is pivotally secured to said scalpel blade via a rod pin that extends through said pivot aperture and said scalpel blade is movably connected to said probe tip housing via a slot pin that extends through said slotted aperture, said slot pin fixedly attached to said probe tip housing.

7. The actuating scalpel device of claim 1 further comprising a handle body and trigger device adapted to be hand held, said driving member extending from said handle body and trigger device through a probe shaft and terminating at said scalpel blade.

8. The actuator scalpel device of claim 7 wherein said handle body and trigger device is adapted to actuate said driving member to move said scalpel blade in and out of said slot.

9. The actuator scalpel device of claim 1 wherein said curved cutting edge is semicircular.

10. A method comprising:
providing an actuating scalpel device that possesses a scalpel blade having a curved cutting edge, a probe tip housing that fully contains said scalpel blade when in a retracted state, a slot in a distal end of said probe tip housing;
deploying said curved cutting edge to extend outside of said probe tip housing via said slot, said scalpel blade moving in an elliptical path defined by a single point on said curved cutting edge traveling from a) when said scalpel blade is in said retracted position to b) when deployed to move outside of said probe tip housing to c) back to said retracted position.

11. The method of claim 10 further comprising controlling said deploying step from a handheld actuating body, wherein extending from said handheld actuating body is an elongated probe that terminates at said probe tip housing.

12. The method of claim 11 wherein said controlling step is accomplished via a trigger adapted to be actuated by a human hand, said trigger extending from said handheld actuating body.

13. The method of claim 11 further comprising moving a driving member in two degrees of freedom with said handheld actuating body, said driving member extending from said handheld actuating body within said elongated probe, said driving member movably pinned to a distal scalpel end of said scalpel blade thereby translating said moving step into degrees of freedom to moving said scalpel blade in said elliptical path.

14. The method of claim 13 wherein said two degrees of freedom is accomplished by rotating gears within said handheld actuating body.

15. The method of claim 13 wherein said driving member possesses a proximal driving member end at said handheld actuating body and a distal driving member end at said distal scalpel end, said proximal driving end and said drive member distal end defining a drive member reference line, said drive member reference line remaining parallel to when said drive member reference line is in said retracted position when in all other positions while carrying out said deployment step.

16. The method of claim 10 wherein said elliptical path is a circular path.

17. The method of claim 10 wherein said curved cutting edge is a semicircle.

18. The method of claim 10 wherein said scalpel blade comprises an elongated element with said curved cutting-edge on a distal end of said elongated element, a pivot aperture located towards a proximal end of said elongated element, and a slotted aperture between said distal end and said proximal end.

19. The method of claim 10 wherein during said deploying step said curved cutting edge rotates in plane with said scalpel blade when moving in said elliptical path.

20. An actuating scalpel device comprising:
a scalpel blade having a noncircular curved cutting edge;
a housing that fully contains said scalpel blade when said scalpel blade is in a retracted state;
a slot in a distal end of said housing through which at least said curved cutting edge is adapted to extend;
a drive member connected to said scalpel blade;
means for actuating said drive member; and
means for driving said scalpel blade to extend out of said slot in an elliptical pathway via said drive member.
